(19) United States
(12) Patent Application Publication
(10) Pub. No.: US 2019/0150757 A1
(43) Pub. Date: May 23, 2019

(54) INFORMATION OBTAINING APPARATUS AND CONTROL METHOD FOR SIGNAL PROCESSING APPARATUS

(71) Applicant: CANON KABUSHIKI KAISHA, Tokyo (JP)

(72) Inventor: Nobuhito Suehira, Tokyo (JP)

(21) Appl. No.: 16/313,784

(22) PCT Filed: Jun. 22, 2017
(86) PCT No.: PCT/JP2017/022972

§ 371 Date: Dec. 27, 2018

(30) Foreign Application Priority Data
Jun. 30, 2016 (JP) 2016-130603

(51) Int. Cl. A61B 5/02; A61B 5/00; A61B 5/145; A61B 5/107

(52) U.S. Cl. CPC A61B 5/02007; A61B 5/0095; A61B 5/743; A61B 5/489; A61B 5/14546; A61B 5/1075; A61B 5/14542

(57) ABSTRACT

An information obtaining apparatus includes a comparison unit configured to compare characteristic information in a first region in an image corresponding to object information with characteristic information in a second region that is different from the first region and a display control unit configured to display information based on a result of the comparison on a display unit.

### TECHNICAL FIELD

The present invention relates to an information obtaining apparatus that obtains object information based on an acoustic wave propagated from an object irradiated with light and a control method for a signal processing apparatus.

### BACKGROUND ART

In recent years, photoacoustic tomography (hereinafter, which will be also referred to as PAT) has been proposed as one of optical imaging technologies. This principle is as follows. First, an object is irradiated with pulsed light, and the light is propagated and diffused in the object. An absorbent in the object absorbs its light energy. The absorbent that has absorbed the light energy performs thermal expansion and generates an acoustic wave. This acoustic wave is referred to as a photoacoustic wave in the present specification. The generated photoacoustic wave is detected by an acoustic wave detecting element. Subsequently, reconstruction processing is performed by using a signal based on the photoacoustic wave detected by the acoustic wave detecting element, and it is possible to obtain a photoacoustic image.

PTL 1 discloses a photoacoustic image apparatus that uses near infrared light. The near infrared light is easily absorbed into blood. For this reason, it is possible to obtain information related to a distribution of blood vessels in the object. In addition, it is possible to obtain information related to an oxygen saturation of the blood vessel from signals obtained by using respective lights of a plurality of wavelengths.

### CITATION LIST

### Patent Literature

PTL 1: Japanese Patent Laid-Open No. 2014-94225

### SUMMARY OF INVENTION

### Technical Problem

The photoacoustic image apparatus can obtain the distribution and oxygen saturation of the blood vessels in the above-described manner. However, the photoacoustic image apparatus does not accurately calculate the oxygen saturation if a background optical coefficient in the object is not specified. Since a living matter is constituted by various tissues such as a skin surface, a fat layer, a muscle layer, and the blood vessels, it is difficult to accurately measure the background optical coefficient.

For this reason, a photoacoustic image apparatus is demanded which can perform a diagnosis support even in a case where the background optical coefficient is not accurately specified. The present invention provides a technique with which a diagnosis by an operator may be supported even in a case where the background optical co-efficient is not accurately obtained.

An information obtaining apparatus that obtains object information based on an acoustic wave propagated from an object irradiated with light according to an aspect of the present invention includes a comparison unit configured to compare characteristic information in a first region in an image corresponding to the object information with characteristic information in a second region that is different from the first region, and a display control unit configured to display information based on a result of the comparison on a display unit.

A control method for a signal processing apparatus according to another aspect of the present invention includes displaying an image of an object based on an acoustic wave propagated from an object irradiated with light on a display unit, and displaying, when a first region and a second region in the displayed image are specified, information based on a comparison of characteristic information in the first region and the second region on the display unit.

Further features of the present invention will become apparent from the following description of exemplary embodiments with reference to the attached drawings.

### BRIEF DESCRIPTION OF DRAWINGS

FIG. 1A is a block diagram illustrating a configuration of a photoacoustic image apparatus according to a first embodiment.

FIG. 1B is a block diagram illustrating a configuration of the photoacoustic image apparatus according to the first embodiment.

FIG. 2 is a flow chart according to the first embodiment.

FIG. 3A is a schematic diagram of a blood vessel according to the first embodiment.

FIG. 3B is a schematic diagram of the blood vessel according to the first embodiment.

FIG. 3C is a schematic diagram of the blood vessel according to the first embodiment.

FIG. 4 is a flow chart for identifying an artery and a vein according to the first embodiment.

FIG. 5A is a flow chart for discriminating a blood vessel type according to the first embodiment.

FIG. 5B is a flow chart for determining the blood vessel type according to the first embodiment.

FIG. 5C is a flow chart for determining the blood vessel type according to the first embodiment.

FIG. 6 illustrates a display example of a diagnostic index according to the first embodiment.

FIG. 7 is a schematic diagram illustrating a blood vessel of a breast according to a second embodiment.

FIG. 8A illustrates a display example of the diagnostic index according to the second embodiment.

FIG. 8B illustrates a display example of the diagnostic index according to the second embodiment.

FIG. 9 illustrates a display example of a circular chart according to the second embodiment.

### DESCRIPTION OF EMBODIMENTS

### First Embodiment

### Photoacoustic Image Apparatus

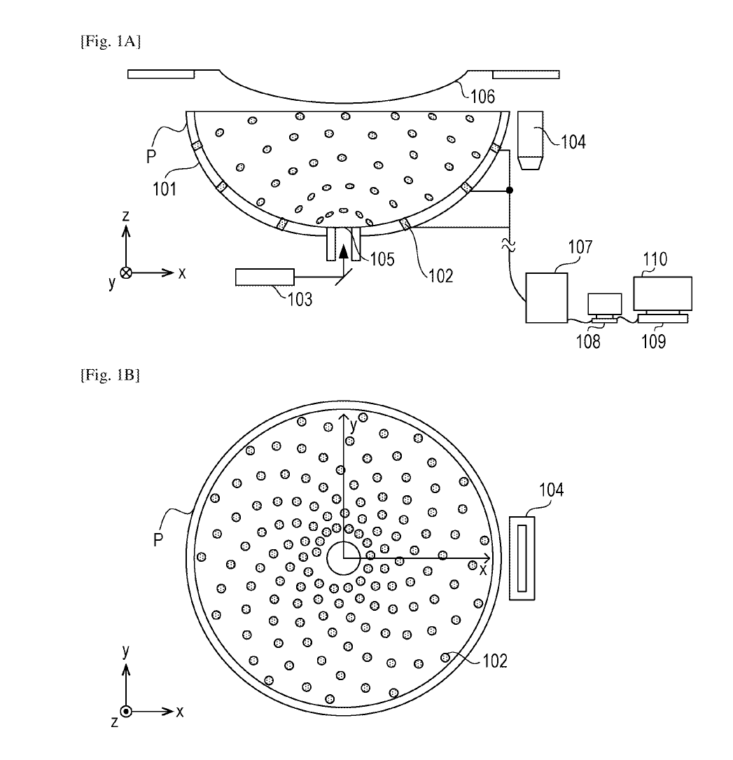

FIGS. 1A and 1B illustrate an information obtaining apparatus according to a first embodiment of the present invention. According to the present embodiment, the information obtaining apparatus is a photoacoustic apparatus using a photoacoustic effect which is configured to obtain information of the inside of the object on the basis of an acoustic wave propagated from an object irradiated with light. FIG. 1A mainly illustrates a cross sectional view of a probe unit of the photoacoustic apparatus and also schematically illustrates parts related to the probe unit and a control unit. FIG. 1B is a plan view while a probe unit P is viewed from the top (z-axis negative direction in the drawing).

The probe unit P includes 512 acoustic wave detecting elements 102 arranged in a spiral manner along an inner surface of a hemispherical container 101 that functions as a supporting member. Furthermore, an opening 105 through which measurement light from a light source unit 103 passes is formed at a bottom part of the container 101. The measurement light is guided to the opening 105 by using a light guiding member such as a mirror or an optical fiber, and the object held by a holding member 106 is irradiated with the measurement light. In the present example, the measurement light is emitted from the z-axis negative direction to a z-axis positive direction. A material such as polyethylene terephthalate having a strength for supporting the object and also having a characteristic for transmitting light and acoustic waves is preferably used for the holding member 106. It should be noted that the inside of the container 101 and the inside of the holding member 106 are filled with an acoustic matching material (for example, water or castor oil) when necessary. It should be noted that the container 101 may have any shape as long as a configuration is adopted in which the acoustic wave detecting elements 102 can be supported such that directions where sensitivities of the plurality of acoustic wave detecting elements is high concentrate in a certain region. For example, the shape does not necessarily need to be hemispherical. The shape may be spherical crown shape or an ellipsoid and also may be a bowl shape formed by connecting a plurality of planes.

A relative positional relationship between the container 101 and the object can be changed by an XY stage which is illustrated in the drawings. For this reason, when the container 101 is moved by the XY stage, the object can be irradiated with pulsed light at a plurality of relative positions with respect to the object. Photoacoustic waves generated from the object at the respective positions are converted into electric signals by the acoustic wave detecting elements 102. The electric signals are stored by a data obtaining unit 107, and reconstruction is performed by a data processing unit 109 by using the stored data, so that it is possible to obtain a three-dimensional photoacoustic image. The data processing unit 109 realizes a function as a comparison unit. Ultrasonic measurement used for form measurement can be performed by an ultrasonic probe 104. The ultrasonic probe 104 has a configuration in which the relative position with respect to the object can be changed by the XY stage together with the container 101. The ultrasonic probe 104 is not limited to a linear type, and configurations appropriate to its use such as a two-dimensional array or a 1.5D array can be used.

A control unit 108 performs instructions related to control on the entire apparatus such as light emission of the light source unit 103, reception control of the data obtaining unit 107, movement of the XY stage, and transmission and reception of the ultrasonic wave by the ultrasonic probe 104. The control unit 108 is provided with a user interface as an input unit configured to accept an input from an operator. The control unit 108 can execute change of a measurement parameter, start and end of a measurement, selection of a processing method for an image, saving of patient information and the image, data analysis, and the like on the basis of the instructions from the operator. Furthermore, detailed data processing is performed by the data processing unit 109, and a photoacoustic image, an oxygen saturation image, and a diagnostic index obtained as the result is displayed on a display unit 110 functioning as a display unit.

### Light Source Unit

A high-power laser light source is preferably used as the light source of the light source unit 103 such that light reaches a deep part of the object. It should be noted however that a light emitting diode, a flash lamp, or the like may also be used. Various types of lasers such as a solid-state laser, a gas laser, a dye laser, and a semiconductor laser can be used as the laser. To effectively generate the photoacoustic wave, the light irradiation is preferably performed in a sufficiently short period of time in accordance with a heat characteristic of the object. For this reason, in a case where the object is a living matter, a pulse width of the pulsed light generated from the light source is preferably approximately 10 to 50 nanoseconds. In addition, a wavelength of the pulsed light is preferably a wavelength at which the light propagates to the inside of the object. Specifically, in the case of the living matter, light having a wavelength of 700 nm or higher but 1100 nm or lower is preferably used.

Herein, a titanium-sapphire laser corresponding to the solid-state laser is used, and light having two wavelengths of 760 nm and 800 nm is used as the measurement light. When the light having the plurality of wavelengths is used, an oxygen saturation can be calculated by using a difference in absorption coefficients for each wavelength. It should be noted that an irradiation timing, the wavelengths, the strength, and the like of the light are controlled by the control unit 108. The laser can alternately emit the light having the two wavelengths respectively at 10 Hz. As a result, the object is irradiated with the light at 20 Hz.

### Acoustic Wave Detecting Element for Photoacoustic Wave

The acoustic wave detecting element 102 is an element configured to receive the photoacoustic wave. Herein, a capacitive micromachined ultrasound transducer (CMUT) is used. The acoustic wave detecting element 102 is a single element having an opening of φ3 mm and a high sensitivity with respect to a band of 0.5 MHz to 5 MHz. Specifically, it means that the sensitivity in the range of 0.5 MHz to 5 MHz of the acoustic wave detecting element becomes a sensitivity that is half or more of the maximum value of the element. Since the band having the high sensitivity includes a low frequency, it is possible to obtain a satisfactory image even in the case of the blood vessel having a thickness of approximately 3 mm. That is, a situation hardly occurs where the inside of the blood vessel is missing and the blood vessel is observed like a ring. 2048 sampling is performed while a sampling frequency is set as 40 MHz. In addition, the data is set as signed 12-bit data.

The signal converted into the electric signal by the acoustic wave detecting elements 102 is transmitted to the data obtaining unit 107, amplified by an amplifier, converted into a digital signal by an analog-to-digital (A/D) converter, and temporarily saved as the data. Thereafter, the data is transmitted to the control unit 108. It should be noted that a reception timing of the acoustic wave is controlled by the control unit 108 so as to be synchronized with the light irradiation.

It should be noted that the acoustic wave detecting element 102 is not limited to the CMUT and may also be constituted by an element using a piezoelectric material.

### Ultrasonic Probe

The ultrasonic probe 104 performs transmission and reception of the ultrasonic wave and can obtain a form image or a Doppler image. Piezoelectric ceramic (PZT) can be used as the element constituting the ultrasonic probe 104. For example, the number of elements is 256, and the band having the sensitivity is 5 MHz to 10 MHz. In addition, 2048 sampling is performed while the sampling frequency is set as 40 MHz.

### Data Processing Unit

The data processing unit 109 generates the photoacoustic image of the inside of the object and the oxygen saturation image corresponding to an image related to the function information (function image) by the image reconstruction using the photoacoustic signal. In addition to the above, desired processing such as light quantity calculation, information processing used for obtaining a background optical coefficient, or signal correction is executed. The data processing unit 109 can be constituted by an information processing apparatus provided with a processor, a memory, and the like. Respective functions of the data processing unit can be realized by respective modules of a program operated by the processor. The data processing unit 109 is a block that realizes a function as the comparison unit. As will be described below, comparison of characteristic information such as an oxygen saturation, a blood vessel diameter, a hemoglobin amount, or a hemoglobin concentration in a plurality of regions is performed. Furthermore, the data processing unit 109 also realizes a function as a display control unit configured to display information based on the comparison of the characteristic information, the photoacoustic image, and the like on the display unit 110.

The photoacoustic image or the oxygen saturation image is displayed on a display corresponding to the display unit 110. A device having a size of 30 inches or larger which can perform color display at a high resolution and having a contrast ratio of 1000:1 or higher is preferably used as the display.

### Image Reconstruction

The image reconstruction is performed by the data processing unit 109. A reconstruction technique such as a universal back-projection method or a phasing addition method is used for the image reconstruction. Herein, a case where the universal back-projection method is used will be described as an example. An initial acoustic pressure distribution P(r) generated by the photoacoustic measurement is represented as Expression 1.

$$P ( r ) = {\int }_{{Ω}_{0}}^{} b ( {r}_{0} , t = \vert r - {r}_{0} \vert ) \frac{d {Ω}_{0}}{{Ω}_{0}} \qquad [ Math . 1 ]$$

A term b(r₀, t) equivalent to projection data at this time is represented as Expression 2. Herein, p_{d}(r₀) denotes a photoacoustic signal detected by the acoustic wave detecting element 102, r₀ denotes positions of the respective detecting elements, t denotes a time, and Ω₀ denotes a solid angle of the acoustic wave detecting element 102. When the data obtained by the data obtaining unit 107 is processed on the basis of Expression 1, it is possible to obtain the initial acoustic pressure distribution P(r).

$$b ( {r}_{0} , t ) = 2 {p}_{d} ( {r}_{0} , t ) - 2 t \frac{\partial {p}_{d} ( {r}_{0} , t )}{\partial t} \qquad [ Math . 2 ]$$

Next, an absorption coefficient distribution can be calculated from the initial acoustic pressure distribution P(r). An acoustic pressure P(r) generated when the absorbent is irradiated with the light is represented as Expression 3.

P(r)=Γ·μₐ(r)·Φ(r) [Math.3]

Γ denotes a Gruneisen coefficient corresponding to an elastic characteristic value.

The Gruneisen coefficient is obtained by dividing a product of a volume expansion coefficient (β) and a square of an acoustic velocity (c) by a specific heat (Cp). μₐ denotes an absorption coefficient at the absorbent. Φ(r) denotes a quantity of light with which the absorbent is irradiated in a local region. It is possible to obtain the absorption coefficient distribution μₐ(r) by solving Expression 3 in terms of the absorption coefficient. It should be noted that the background optical coefficient does not appear in the absorption coefficient distribution since the background optical coefficient is sufficiently lower than the absorption coefficient of the absorbent.

The light quantity Φ(r) can be represented by using a variable z as in Expression 4, for example, in a case where the light quantity uniformly attenuates in a depth direction.

Φ=Φ₀EXP(−μ_{eff}z) [Math.4]

Φ₀ denotes a light quantity of incident light on the surface. μ_{eff} denotes an average equivalent damping coefficient in the object and reflects a background scattering coefficient μ_{bs} in the object and an absorption coefficient μ_{ba}. For example, the average equivalent damping coefficient is represented as Expression 5.

μ_{eff}=\sqrt{3μ_{ba}μ_{bs}} [Math.5]

It should be noted that the background scattering coefficient in the object and the absorption coefficient can be measured, for example, by a near infrared spectrometry or the like. In the near infrared spectrometry, the object is irradiated with the light, and the light from the object is received. Subsequently, a waveform of the signal is analyzed by a time domain or a frequency domain to obtain the background optical coefficient. These apparatuses perform the analysis by using a homogeneous medium as a model. For this reason, it is difficult to accurately measure the background optical coefficient of the living matter constituted by various tissues such as a skin surface, a fat layer, a muscle layer, and the blood vessels.

Next, a method of obtaining a hemoglobin distribution from the absorption coefficient distribution will be illustrated. The absorption coefficient μa(λ) of the absorbent is determined by absorption of oxidized hemoglobin CHbO and reduced hemoglobin CHbR per unit volume. When absorption coefficients of the oxidized hemoglobin and the reduced hemoglobin are set as EHbO(λ₁), EHbR(λ₁), EHbO(λ₂), and EHbR(λ₂) at respective wavelengths, the absorption coefficients are represented as Expression 6. It should be noted that the left term is the absorption coefficient distribution obtained by Expression 3.

μₐ(λ₁)=ε_{HbO}(λ₁)C_{HbO}+ε_{HbR}(λ₁)C_{HbR}

μₐ(λ₂)=ε_{HbO}(λ₂)C_{HbO}+ε_{HbR}(λ₂)C_{HbR} [Math.6]

When deformation from Expression 6 with regard to the oxidized hemoglobin and the reduced hemoglobin is performed, the oxidized hemoglobin and the reduced hemoglobin are respectively represented as Expression 7.

$${C}_{HbO} = \frac{{ɛ}_{HbR} ( {λ}_{2} ) {μ}_{a} ( {λ}_{1} ) - {ɛ}_{HbR} ( {λ}_{1} ) {μ}_{a} ( {λ}_{2} )}{{ɛ}_{HbO} ( {λ}_{1} ) {ɛ}_{HbR} ( {λ}_{2} ) - {ɛ}_{HbR} ( {λ}_{1} ) {ɛ}_{HbO} ( {λ}_{2} )} \\ {C}_{HbR} = \frac{{ɛ}_{HbO} ( {λ}_{1} ) {μ}_{a} ( {λ}_{2} ) - {ɛ}_{HbO} ( {λ}_{2} ) {μ}_{a} ( {λ}_{1} )}{{ɛ}_{HbO} ( {λ}_{1} ) {ɛ}_{HbR} ( {λ}_{2} ) - {ɛ}_{HbR} ( {λ}_{1} ) {ɛ}_{HbO} ( {λ}_{2} )} \qquad [ Math . 7 ]$$

It should be noted that, since the total hemoglobin (tHb) is a total amount of the oxidized hemoglobin and the reduced hemoglobin, the total hemoglobin is represented as Expression 8.

tHb=C_{HbO}+C_{HbR} [Math.8]

Since an oxygen saturation StO is a ratio of the oxidized hemoglobin in the total hemoglobin, the oxygen saturation is represented as Expression 9.

$$StO = \frac{{C}_{HbO}}{{C}_{HbR} + {C}_{HbO}} \qquad [ Math . 9 ]$$

### Regarding Background Optical Coefficient

Here, an example of an experiment using a phantom will be described with regard to an influence in a case where the background optical coefficient is different from a true value. As the phantom, nylon wires of φ1 mm respectively arranged at depths of 10 mm and 20 mm as targets in urethane resin having the scattering coefficient of 0.8/mm and the absorption coefficient of 0.004/mm comparable with values of human mammary gland tissues are used. One nylon wire imitates the oxygen saturation of an artery (for example, 95%), and the other nylon wire imitates the oxygen saturation of a vein (for example, 80%). Adjustment of the oxygen saturation can be realized by using a plurality of color materials having different absorption coefficients depending on wavelengths. The absorption coefficient of the nylon wire is set as 0.1/mm at the wavelength of 800 nm comparable with blood.

In this case, if the value obtained by measuring the background optical coefficient in the urethane resin varies from the true value by 5%, the oxygen saturation at the depth of 20 mm may vary by approximately 10%. As illustrated in Expression 4, since the error of the oxygen saturation depends on the depth, the error becomes larger as the depth becomes deeper. Therefore, in a case where the background optical coefficient has the error, the numeric value calculated as the oxygen saturation StO may exceed 100%.

On the other hand, in the case of the target imitating the oxygen saturation of the artery and the target imitating the oxygen saturation of the vein located at equivalent depths, a result obtained by dividing the oxygen saturations of those tends to have a constant value calculated irrespective of the depths where the two targets are arranged. It is conceivable that this is because the errors in the respective wavelengths are cancelled by the division, and the influence becomes smaller.

### Measurement and Signal Processing

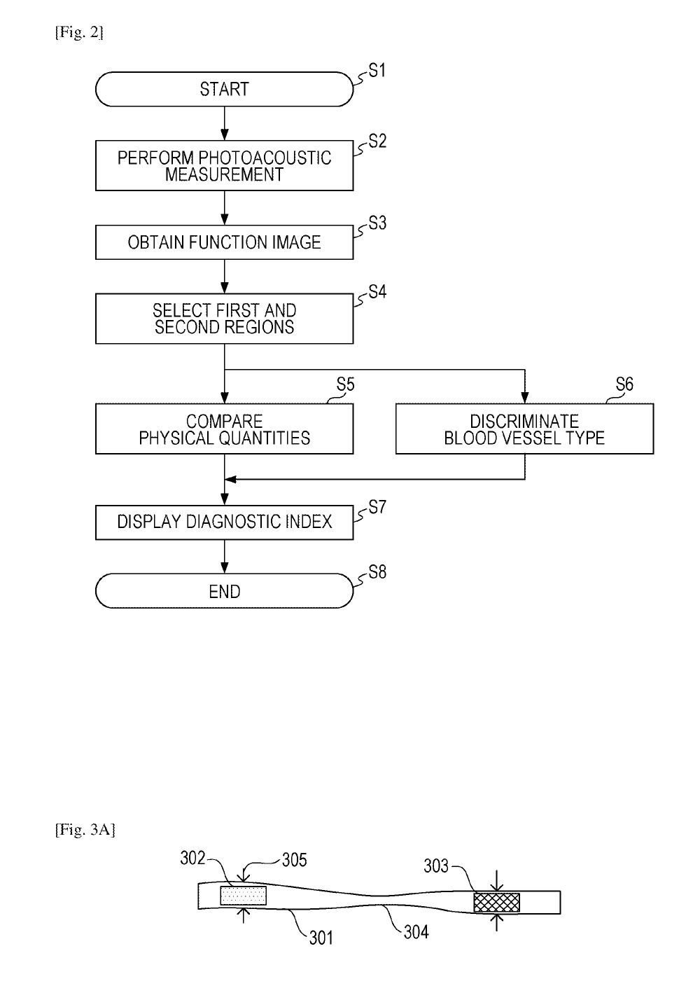

FIG. 2 illustrates a flow chart for displaying the diagnostic index related to the processing according to the present embodiment.

In step S1, the measurement is started. In this state, the object is inserted into the holding member 106 so as to be contacted with each other. The holding member 106 and the object are in close contact such that air does not exist between the holding member 106 and the object, and the surrounding of the object is filled with water corresponding to acoustic matching liquid. It should be noted that the ultrasonic wave measurement may be performed by the ultrasonic probe 104 before the photoacoustic image is obtained. This is because coordinates of a lesioned part of the object or the like are identified by the ultrasonic wave image.

In step S2, the photoacoustic measurement is performed. First, an operator issues a measurement instruction from the control unit 108. As a result, the container 101 is moved to a desired position by the XY stage. Subsequently, the pulsed light is emitted from the light source unit 103, and in synchronism with this, the acoustic wave detecting element 102 receives the photoacoustic wave. With regard to the irradiation of the pulsed light, while the container 101 is moved in a spiral manner, the irradiation of the light having the two waveforms of 760 nm and 800 nm is alternately performed. Finally, the photoacoustic waves are obtained at the respective wavelengths at a position of 1024. Imaging ranges can be set as diameters of 80 mm, 120 mm, and 160 mm by selection. It should be noted that the influence of the positional shift between the wavelengths due to a body motion or the like becomes smaller when the light having the different wavelengths is alternately emitted to obtain the photoacoustic image for the two wavelengths as compared with a case where the measurement is performed throughout at one wavelength, and thereafter the measurement is performed at the other wavelength. That is, the above-described control method is more preferably used to calculate the oxygen saturation.

In step S3, the function image is generated from the photoacoustic image. The image reconstruction is performed from the obtained data, and the initial acoustic pressure distribution for each wavelength is obtained. Subsequently, the absorption coefficient distribution can be obtained by using the above-described expressions. The light quantity distribution can be obtained by using the optical coefficient at which the object is measured by the near infrared spectrometry and form information of the holding member 106. It should be noted that the data of the ultrasonic probe 104 may also be used as the form information. Thereafter, the oxygen saturation image corresponding to the function image is obtained. With regard to the oxygen saturation, the oxygen saturation may be calculated in a part where the absorption coefficient is higher than or equal to a threshold in the absorption coefficient distribution instead of the calculation over the specified entire region. That is, since the absorption coefficient at the position where the blood vessel exists is higher than the absorption coefficient at the position where the blood vessel does not exist, it is possible to selectively display only the oxygen saturation at the position of the blood vessel.

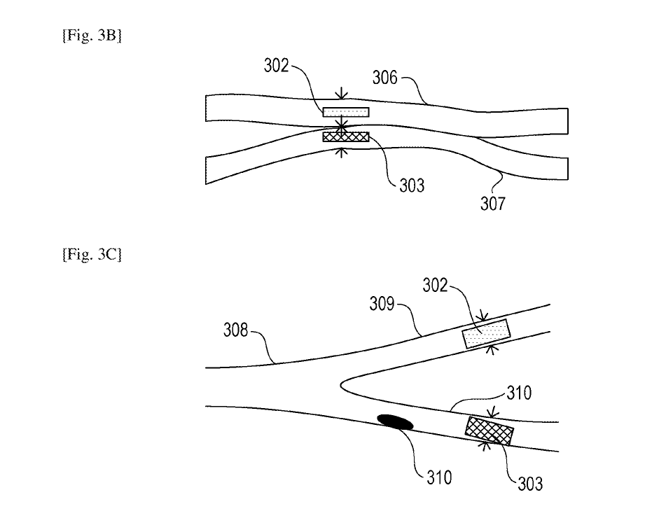

In step S4, a first region and a second region are selected. The operator uses, for example, a user interface of the control unit 108 on a screen to select the first region and the second region from the oxygen saturation image corresponding to the function image displayed on the display unit 110. FIGS. 3A to 3C illustrate examples in which parts of the blood vessel in the function image are set as the first region and the second region. FIG. 3A illustrates a narrowed part 304 of the blood vessel. The example is illustrated in which a first region 302 and a second region 303 are set in different positions of this blood vessel (that is, an upstream side and a downstream side of the narrowed part). In FIG. 3B, the first region 302 is set in one of accompanying artery 306 and vein 307, and the second region 303 is set in the other of the accompanying artery 306 and vein 307. In FIG. 3C, with regard to the blood vessel 308 having branches, the first region 302 is set in one of the branches, and the second region 303 is set in the other of the branches. A branched part where the second region 303 is set has the blocked part 310. It should be noted that, in the respective drawings of FIGS. 3A to 3C, the first and second regions are illustrated as rectangular areas in the blood vessel, but the specifications of the respective regions are not limited to this technique. For example, when the operator specifies a region wider than the width of the blood vessel, the data processing unit 109 may extract a part equivalent to the blood vessel in the region to be set as the first region or the second region. As another method, when the operator specifies an arbitrary position in the blood vessel, a predetermined range in the blood vessel including the specified position may be set as the first region or the second region. Furthermore, as still another example, the data processing unit 109 may extract and automatically set parts before and after the narrowed part of the blood vessel, the accompanying blood vessel, or the branch of the blood vessel by a technique such as pattern matching, or present the extracted regions to the operator and ask the operator to select the first and second regions.

In step S5, comparison of the characteristic information in the first region 302 and the second region 303 is performed. Examples of the characteristic information include the oxygen saturation, the total hemoglobin amount, the blood vessel diameter, and the like. The oxygen saturation and the total hemoglobin amount are average values of the selected parts. The blood vessel diameter 305 is an average value of the blood vessel diameters where the selected regions are included. The comparison of the characteristic information is performed by dividing the characteristic information in the first region by the characteristic information in the second region. It should be noted that the comparison is not limited to the division and can also be realized by the subtraction. However, as described above, the division is preferably performed since the error of the depth direction can be cancelled.

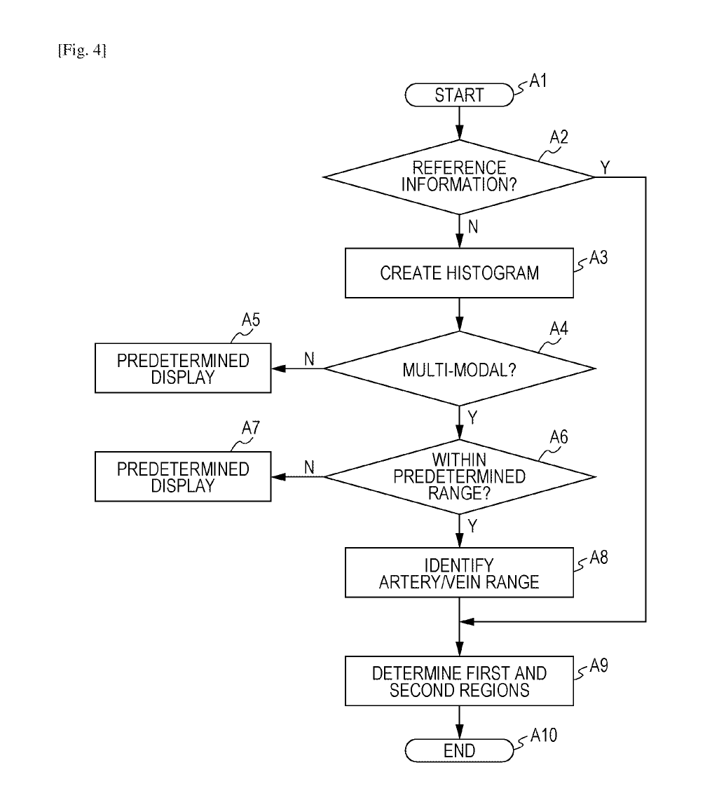

In step S6, a type of the blood vessel in the first region 302 and the second region 303 is discriminated. The type of the blood vessel refers to types such as the artery and the vein. Hereinafter, a flow for determining the type of the blood vessel will be described with reference to FIG. 4. It should be noted that the processing in step S6 may be executed in parallel with step S5.

In step A1, processing of determining the type of the blood vessel is started.

In step A2, the presence or absence of reference information is determined. The reference information includes finding by a doctor, anatomical structural information detected by the pattern matching or the like performed by the data processing unit 109, a result of Doppler measurement by the ultrasonic apparatus, and the like. In a case where the reference information is present, the flow proceeds to step A9, and the determination on the type of the blood vessel is performed. On the other hand, in a case where the input of the reference information is absent, the flow proceeds to step A3, and the processing of determining the type of the blood vessel is performed by using the oxygen saturation image obtained from the photoacoustic image apparatus. The oxygen saturation image is three-dimensional data including a desired blood vessel of FIGS. 3A to 3C in a range of 30 mm×30 mm×10 mm, for example. This range may be specified by the operator or may be automatically set by the data processing unit 109. In a case where the specified range is not sufficiently large, there is a fear that a reliability of a histogram generated in step A3 may not be sufficient. For this reason, the data processing unit 109 can also support the operator such that the sufficient reliability of the histogram generated in the range set by the operator can be attained. For example, in a case where the range specified by the operator is narrower than a predetermined value, the range may be automatically expanded so that the range is not set to be narrower than the predetermined value.

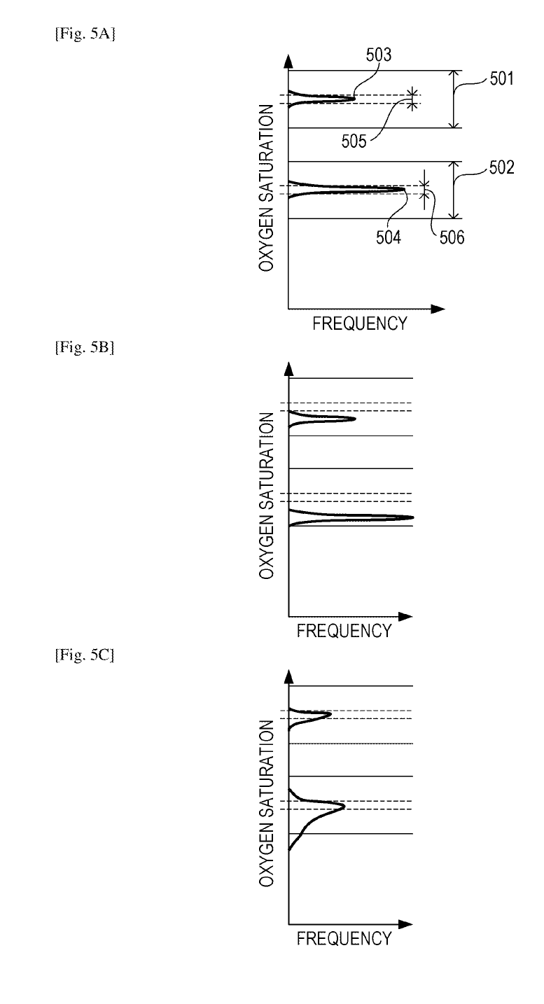

In step A3, the data processing unit 109 generates the histogram of the oxygen saturation from the oxygen saturation image. FIGS. 5A to 5C schematically illustrate the histogram of the oxygen saturation. The vertical axis represents the oxygen saturation, and the horizontal axis represents the frequency. FIG. 5A illustrates an example in a case where the oxygen saturation is accurately calculated. In this example, two peaks of the frequency appear in positions having different oxygen saturations. It is conceivable that first peak 503 in the position having the higher oxygen saturation is derived from the artery, and a second peak 504 in the position having the lower oxygen saturation is derived from the vein. FIG. 5B illustrates a case where the oxygen saturation is calculated from a region at a certain depth from the surface. Since the background optical coefficient is deviated from the actual value, the oxygen saturation indicates a frequency distribution different from the frequency distribution illustrated in FIG. 5A.

FIG. 5C illustrates a state in which a normal oxygen saturation and an abnormal oxygen saturation exist in a mixed manner. An example of the abnormal oxygen saturation includes a case where oxygen supply from the blood vessel becomes insufficient in the tumor part, and a hypoxic state is established. In a case where the blood vessel is clogged because of narrowing or the like, the oxygen saturation may be decreased, and a frequency distribution as illustrated in FIG. 5C may be obtained.

In step A4, a determination on whether or not the histogram is multi-modal. In a case where the normal artery and vein are included in the specified region, the histogram becomes bimodal having at least two peaks. In a case where the blood vessel in which the oxygen saturation is decreased by the tumor or the occlusion exists, the number of peaks may be further increased. For this reason, since it is conceivable that the oxygen saturation can be normally calculated when the histogram is multi-modal, the flow proceeds to step A6. On the other hand, in the case of a unimodal histogram, the flow proceeds to step A5.

In the case of the unimodal histogram, a case where the blood vessel is not present in the measurement range, a case where only one of the artery and the vein is present, and the like are conceivable. In view of the above, according to the present embodiment, in a case where the histogram is unimodal in step A4, the operator is notified in step A5 that the reliability of the obtained measurement result is low. After the notification to the operator is performed, the processing may be ended. After the notification for urging the operator to set the measurement range again is performed, the processing in step A1 and subsequent steps may also be performed.

In a case where it is determined in step A4 that the histogram is multi-modal, it is determined in step A6 whether or not the oxygen saturation in the histogram is within a predetermined range. When the oxygen saturation is within the predetermined range, the first peak 503 of the histogram is within a 20% range 501 where a normal range 505 of the oxygen saturation of the artery (for example, 93 to 99%) is set as a center, and the second peak 504 of the histogram is within a 20% range 502 where a normal range 506 of the oxygen saturation of the vein (for example, 73 to 77%) is set as a center. When the first peak 503 and the second peak 504 are within the predetermined ranges, it is conceivable that the oxygen saturation can be normally calculated, and the flow proceeds to step A8. On the other hand, in a case where at least one of the first peak 503 and the second peak 504 is not within the predetermined range, the flow proceeds to step A7.

It should be noted that the function information including the oxygen saturation is more accurately calculated as the position is closer to the surface of the object. For this reason, in a case where it is determined that the value is not within the setting value in the used range, whether or not the value is within the set value in the range including the data in the vicinity of the surface may be checked. In a case where the same segments exist in left and right, a normal segment may be measured to check whether or not the value is within the setting value. With this configuration, it is possible to distinguish whether or not the value of the optical coefficient has an error. It should be noted that the range of the setting value may be changed in accordance with the depth.

In step A7, predetermined display is performed. As a case where the oxygen saturation is not within the predetermined range, there is a possibility that the optical coefficient is not an accurate value. In a case where the normal site is within the setting value and a target site is not within the setting value, cyanosis or the like is conceivable. For example, the oxygen saturation of the artery of central cyanosis is 82% or lower, and the oxygen saturation of the vein is 52% or lower, for example. In addition, the oxygen saturation of the artery of peripheral cyanosis is a normal value, and the oxygen saturation of the vein is 33% or lower, for example. Furthermore, local peripheral circulatory insufficiency may be generated due to arterial occlusive disease or venous occlusive disease. Type of possible diseases as described above may be displayed.

In step A8, the range of the artery and the vein is identified. It is determined in which region in the function image a group where the first region and the second region include the first peak 503 and a group where the first region and the second region include the second peak 504 are included. In the case of the region corresponding to the group including the first peak 503, a probability is high that this is the artery. In the case of the region corresponding to the group including the second peak 504, a probability is high that this is the vein.

In step A9, a determination is performed on which one of the artery and the vein the first region and the second region selected in step S4 respectively correspond to. The data processing unit 109 determines which one of the first region and the second region the first region and the second region respectively correspond to on the basis of the range of the artery and the vein identified in step S8.

In step A10, the process flow for determining the type of the blood vessel is ended, and the process flow proceeds to step S7 of FIG. 2.

In step S7, the diagnostic index that takes into account the comparison result of the characteristic information obtained in step S5 and the discrimination result of the type of the blood vessel obtained in step S6 as described above is displayed. As illustrated in FIG. 6, the type of the blood vessel, the oxygen saturation, the total hemoglobin amount, the blood vessel diameter, and the like are respectively displayed with respect to a region 1 and a region 2 as the diagnostic index, and furthermore, those comparison results are displayed. The hemoglobin concentration in the relevant region may be indicated instead of the total hemoglobin amount or together with the total hemoglobin amount. The comparison result is a ratio of numeric values in the respective regions, for example. The diagnosis can be supported by displaying these items.

For example, when attention is focused on the oxygen saturation, since the oxygen saturations of the first region 302 and the second region 303 in the same blood vessel should be matched with each other in a normal state, a ratio of those is 1. However, in a case where the oxygen saturations are varied in the parts before and after the narrowed part 304 as illustrated in FIG. 3A, if the ratio of the oxygen saturations is not in a range between 0.98 and 1.02, for example, the blocking or narrowing of the blood vessel is suspected. The range between 0.98 and 1.02 is set as values that take the error into account. When attention is focused on accompanying artery/vein as illustrated in FIG. 3B, the ratio of the oxygen saturation of the artery to the oxygen saturation of the vein in a normal state is 1.24 to 1.32. When a result obtained by dividing the value of the artery by the value of the vein is lower than 1.24, it is conceivable that the oxygen saturation of the artery has an abnormality, and the blocking or narrowing of the artery is suspected. On the other hand, when the ratio is higher than 1.32, the hypoxic state is established, and the tumor or narrowing is suspected. The same also applies when attention is focused on the blood vessel that is not the accompanying blood vessel. In FIG. 3C, the same blood vessel is branched. Therefore, the ratio of the oxygen saturations of the different branches becomes 1 in a normal state. Similarly as in the case of FIG. 3A, while the error is taken into account, if the ratio of the oxygen saturation of the first region to the oxygen saturation of the second region is not the range between 0.98 and 1.02, the blocking or narrowing of the blood vessel is suspected. In this manner, when the comparison of the characteristic information of the different regions is performed, it is possible to support the diagnosis by the operator.

Attention is focused on the hemoglobin concentration. When the hemoglobin concentration is high, an increased viscosity of the blood is suspected. When the hemoglobin concentration is low, oxygen deficiency is suspected. Attention is focused on the blood vessel thickness. In a case where a result is obtained that the artery is narrow by the comparison of the blood vessel diameters of the artery and the vein in the accompanying part, the blocking or narrowing of the artery is suspected. It should be noted that the comparison may also be displayed in each of the normal part and the target part. The comparison may also be performed on the basis of the area or volume of the blood vessel other than the blood vessel thickness, that is, the blood vessel diameter.

The predetermined range is previously stored in a storage unit of the data processing unit 109 with regard to the comparison result of various characteristic information. In a case where the comparison result obtained as the result of the measurement is not within the predetermined range, the display is preferably performed to facilitate the recognition of the operator while a color of a column corresponding to this comparison result is set to be different from a color of the other column, a size or type of a font is set to be different from that of the other column, or blinking is performed for the display. That is, the information can be emphasized while the comparison result is displayed by different display methods in a case where the comparison result satisfies a predetermined condition and a case where the comparison result does not satisfy the predetermined condition. Furthermore, in a case where the comparison result is not within the predetermined range, a name of a disease that may be suspected from the comparison result can be presented. That is, the diagnosis by the operator is supported by presenting the disease name corresponding to the comparison result.

The data processing unit 109 may also display the comparison result on the display unit 110 together with the function image. When the operator specifies the first region and the second region from among the function images while observing the function image illustrated in FIGS. 3A to 3C, the comparison result of FIG. 6 may be updated in accordance with the specification. Furthermore, a third region different from the first region and the second region may be specified to present the comparison result of the characteristic information in the respective regions to the operator.

As described above, according to the present embodiment, even if the background optical coefficient is not an accurate value, the information useful to the diagnosis on the presence or absence of the tumor, the artery obstruction, the venous obstruction, or the like is provided by displaying the information of the type of artery/vein and the comparison result of the characteristic information, and the diagnosis can be supported.

### Second Embodiment

Another embodiment of the present invention will be described. Descriptions will be omitted with regard to aspects common to the first embodiment, and different aspects will be mainly described.

According to the first embodiment, the first region and the second region are set in the specific blood vessel. However, the method for the setting of the respective regions is not limited to this, and the first region and the second region may be regions including a plurality of blood vessels.

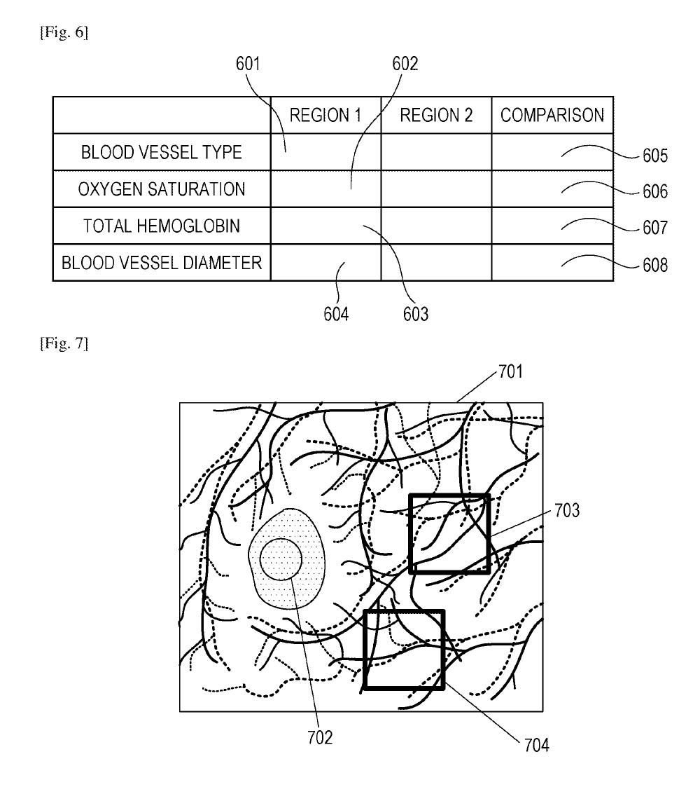

FIG. 7 is a schematic diagram of an oxygen saturation image 701 of a breast in a range including a nipple 702. The artery is indicated by a solid line, and the vein is indicated by a dotted line. A state is illustrated in which a first region 703 and a second region 704 are set in the oxygen saturation image 701. It can be checked that the artery and the vein run through while the nipple 702 is set as a center. It should be noted that, according to the present embodiment, the nipple 702 is illustrated for convenience of the descriptions, but the nipple 702 does not appear in an image depending on a manner of displaying the image (such as a range of a depth direction to be displayed).

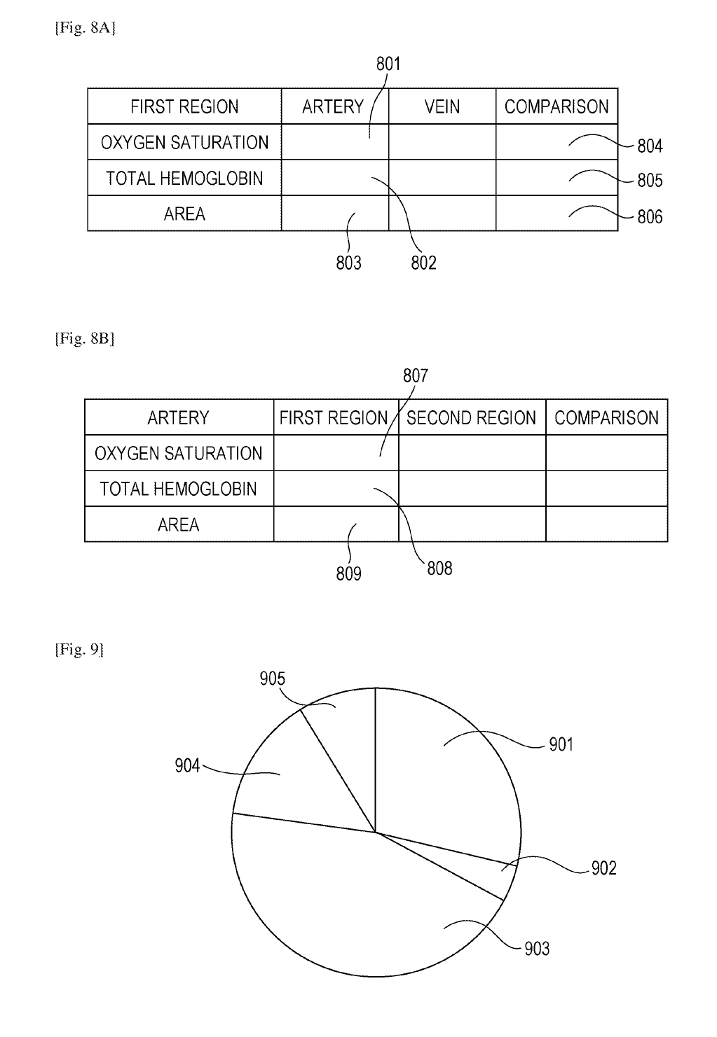

According to the present embodiment, FIGS. 8A and 8B illustrate an example of a table presented as the information based on the comparison result. FIG. 8A illustrates the further comparison of the characteristic information included in the region of the artery and the region of the vein in the first region 703. In this example, the table is constituted by the oxygen saturation, the total hemoglobin amount, and the volume. An average value in the region is calculated for the oxygen saturation and the total hemoglobin amount. It should be noted that the hemoglobin concentration of this region may be indicated instead of the total hemoglobin amount or together with the total hemoglobin amount. The volume is calculated by counting the number of pixels of the artery or the vein. It should be noted that the discrimination of the artery and the vein can be performed by using the technique described according to the first embodiment. The group determined as the artery by the histogram and the group determined as the vein are respectively displayed, and with this configuration, it is possible to perform the comparison of the artery and the vein in the same region. With regard also to the second region 703, it is possible to present the similar information.

FIG. 8B illustrates the result of the mutual comparison of the arteries in the first region 703 and the second region 704. Similarly, FIG. 8B illustrates the result of the mutual comparison of the veins in the first region 703 and the second region 704. With this configuration, it is possible to perform the mutual comparison of the arteries or the mutual comparison of the veins in the different regions.

The first region 703 and the second region 704 do not necessarily need to be the photoacoustic image obtained by the single measurement. For example, in the case of the breast, the first region may be set in the right breast, and the second region may be set in the left breast. If the tumor, aneurysm, varicose, or the like exists in one of the regions, the comparison result of the numeric values in the first region and the second region may indicate a peculiar value. Since the characteristic information at the equivalent depths can be compared, it is effective to detect the abnormality.

In addition, the ratio of the oxygen saturations in the first region and the second region in the vicinity of the surface or the ratio of the oxygen saturations in the first region and the second region in the vicinity of the desired depth may be displayed. As already described above, since the ratio of the oxygen saturations tends to be constant irrespective of the depth, it is effective to detect the abnormality.

It should be noted that a circular chart as illustrated in FIG. 9 may be used as the display method for the numeric values in the respective regions. For example, with regard to the blood vessel included in the first region, FIG. 9 can indicate proportions respectively occupied by the artery 901 (equivalent to the range 505 in FIG. 5A) indicating the normal oxygen saturation, the artery 902 (equivalent to the part except for the range 501 to the range 505 in FIG. 5A) indicating the value outside of the range of the normal oxygen saturation, the vein 903 (equivalent to the range 506 in FIG. 5A) indicating the normal oxygen saturation, the vein 904 (equivalent to the part except for the range 502 to the range 506 in FIG. 5A) indicating the value outside of the range of the normal oxygen saturation, and the other 905 (equivalent to the part except for the range 501 and the range 502 in FIG. 5A). When respective circular charts in the first region and the second region are compared with each other, it may be possible to visually find out differences in the proportions. Furthermore, a division image obtained by division of the oxygen saturation of the vein with respect to the artery at the same depth may be newly created and displayed.

As described above, according to the present embodiment, even if the background optical coefficient is not an accurate value, the information useful to the diagnosis is provided, and the diagnosis can be supported.

### Third Embodiment

Still another embodiment of the present invention will be described. Descriptions will be omitted with regard to aspects common to the above-described embodiments, and different aspects will be mainly described.

According to the present embodiment, in accordance with the specification of a certain region in the object by the operator of the photoacoustic image apparatus, the comparison of the characteristic information of the artery and the vein in the specified region is performed. For example, the operator specifies the region in a state in which the oxygen saturation image 701 is presented as illustrated in FIG. 7. The photoacoustic image apparatus extracts the artery and the vein included in the specified region in response to this specification and executes the comparison processing of the characteristic information of the artery and the vein in this region to display the result on the display unit. The technique described according to the first embodiment can be used for the discrimination on the artery and the vein. In addition, the characteristic information to be compared is also as described above.

According to the first embodiment, the operator selects the first region 302 and the second region 303 in the example described with reference to FIG. 3B. In contrast to this, according to the present embodiment, when the operator specifies the region including the target artery and vein without individually specifying the blood vessel, the photoacoustic image apparatus discriminates the artery and the vein included in the region and executes the comparison processing of the characteristic information of the artery and the vein. For this reason, it is more facilitated for the operator to obtain the comparison result of the characteristic information.

It should be noted that the characteristic information and the comparison method illustrated according to the above-described respective embodiments are merely for exemplifications and can be modified in any forms in a range without departing from the technical idea of the present invention. For example, according to the respective embodiments, the case has been described where the first region and the second region are specified from the oxygen saturation image corresponding to the function image, but the first and second regions may also be specified from the initial acoustic pressure distribution image or the absorption coefficient distribution image obtained by using light having one wavelength. That is, the first and second regions may be specified from any image as long as the image corresponding to the object information.

In addition, the descriptions have been given while the blood vessel is used as the example according to the above-described respective embodiments, but the present invention can also be applied to structures other than the blood vessel.

According to the embodiments of the present invention, even in a case where the background optical coefficient of the object is not accurately obtained, it is possible to perform the diagnosis support.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

This application claims the benefit of Japanese Patent Application No. 2016-130603, filed Jun. 30, 2016, which is hereby incorporated by reference herein in its entirety.

1. An information obtaining apparatus that obtains object information based on an acoustic wave propagated from an object irradiated with light, the information obtaining apparatus comprising:
a comparison unit configured to compare characteristic information in a first region in an image corresponding to the object information with characteristic information in a second region that is different from the first region; and
a display control unit configured to display information based on a result of the comparison on a display unit.

2. The information obtaining apparatus according to claim 1, wherein the characteristic information in the first region and the characteristic information in the second region are function information of the object.

3. The information obtaining apparatus according to claim 1, wherein the first region and the second region are blood vessels of the object.

4. The information obtaining apparatus according to claim 3, wherein the first region and the second region correspond to different parts of the same blood vessel.

5. The information obtaining apparatus according to claim 3, wherein one of the first region and the second region is an artery and the other one is a vein.

6. The information obtaining apparatus according to claim 5, wherein the comparison unit further determines types of the blood vessels in the first region and the second region, and the display control unit further displays the types of the blood vessels in the first region and the second region on the display unit.

7. The information obtaining apparatus according to claim 6, wherein the comparison unit determines the types of the blood vessels in the first region and the second region on the basis of a histogram of the characteristic information in the first region and the second region.

8. The information obtaining apparatus according to claim 3, wherein the first region and the second region are the blood vessels located at equivalent depths.

9. The information obtaining apparatus according to claim 1, wherein the characteristic information in the first region and the characteristic information in the second region are at least one of oxygen saturations and hemoglobin amounts in the respective regions, and a length, an area, and a volume of a structure in the object.

10. The information obtaining apparatus according to claim 1, wherein the display control unit displays plural pieces of characteristic information with regard to the first region and the second region on the display unit.

11. The information obtaining apparatus according to claim 1, wherein the display control unit displays a type of a disease on the display unit as the information based on the result of the comparison.

12. The information obtaining apparatus according to claim 1, further comprising an input unit configured to accept specifications of the first region and the second region from the object information.

13. A control method for a signal processing apparatus that processes a signal based on an acoustic wave propagated from an object irradiated with light, the control method comprising:
displaying an image of the object based on the acoustic wave on a display unit; and
displaying, when a first region and a second region in the displayed image are specified, information based on a comparison of characteristic information in the first region and the second region on the display unit.

14. The control method for the signal processing apparatus according to claim 13, wherein the image of the object is an image indicating function information of the object.

15. The control method for the signal processing apparatus according to claim 13, wherein the first region and the second region are blood vessels, the control method further comprising displaying of types of the blood vessels in the first region and the second region.

16. The control method for the signal processing apparatus according to claim 13, further comprising accepting the specifications of the first region and the second region in the image.

17. The control method for the signal processing apparatus according to claim 13, further comprising varying the display in a case where the information based on the comparison of the characteristic information is not within a range of a predetermined value from the display in a case where the information is within the range of the predetermined value.

18. The control method for the signal processing apparatus according to claim 13, wherein a type of a disease is displayed on the display unit as the information based on the comparison in the displaying the information based on the comparison.

19. The control method for the signal processing apparatus according to claim 13, wherein, in a case where the result of the comparison satisfies a predetermined condition, a part where the predetermined condition is satisfied and the other part are displayed by using different display methods on the display unit in the displaying the information based on the comparison.

20. The control method for the signal processing apparatus according to claim 13, wherein the characteristic information in the first region and the characteristic information in the second region are at least one of oxygen saturations and hemoglobin amounts in the respective regions, and a length, an area, and a volume of a structure in the object.
